(19) United States
(12) Patent Application Publication
(10) Pub. No.: US 2007/0179573 A1
(43) Pub. Date: Aug. 2, 2007

(54) Method and apparatus for stimulating hair growth

(76) Inventor: Richard Laurent, Bloomfield Township, MI (US)

Correspondence Address: RADER, FISHMAN & GRAUER PLLC, 39533 WOODWARD AVENUE, SUITE 140, BLOOMFIELD HILLS, MI, 48304-0610, US

(21) Appl. No.: 11/701,600

(22) Filed: Feb. 2, 2007

Related U.S. Application Data
(60) Provisional application No. 60/764,471, filed on Feb. 2, 2006.

(51) Int. Cl. A61N 5/06

(52) U.S. Cl. 607/89

(57) ABSTRACT

A laser device for stimulating a body region to promote hair growth is disclosed. The laser device includes a housing structure, a plurality of pins, a plurality of laser modules, and a microcontroller. The housing structure includes a removable head assembly. The pins are configured to stimulate the body region. The laser modules are configured to provide laser energy to the body region. The microcontroller is configured to control the plurality of laser modules at a selected power, pulse levels and pulse durations. The pins and lasers are mounted through holes in the housing structure.

### BACKGROUND

There are many known methods for treating thinning hair or alopecia (human hair loss) including topical creams and surgical implants. One of the most recent developments is the use of Low Level Laser Therapy (LLLT). The basic principle behind LLLT is that light can affect cell functions by increasing blood and lymph circulation to the hair roots, removing blockages around the hair bulb, and energizing the hair root to take in nutrients faster.

### BRIEF DESCRIPTION OF THE DRAWINGS

FIG. 1 illustrates a front view of a laser device according to an embodiment;

FIG. 2 illustrates a side view of the laser device according to FIG. 1;

FIG. 3 illustrates a side internal view of the laser device according to FIGS. 1 and 2;

FIG. 4 illustrates an exemplary schematic of the laser device according to an embodiment;

FIG. 5 shows a matrix illustrating power consumption data for a laser device having three laser modules;

FIG. 6 shows a matrix illustrating power consumption data for a laser device having five laser modules;

FIG. 7 shows a matrix illustrating power consumption data for a laser device having nine laser modules; and

FIG. 8 is a flow chart illustrating the functionality of a laser device according to an embodiment.

### DETAILED DESCRIPTION OF THE INVENTION

A method and apparatus for the application of stimulating hair growth (hair Rejuvenator module) using a hand held, cordless, battery operated device is provided. The device includes a plurality of laser modules configured to stimulate the hair, follicles, papilla, and surrounding tissue cells of a body region by providing free space coherent light energy in the form of low level laser therapy (LLLT). Body regions may include, but are not limited to, head, arms, legs, and chest. However, a common region for stimulating hair growth is the head, where the treatment region includes the hair, follicles, skin and scalp. The device further includes a plurality of pins selectively positioned with respect to each laser module to provide further hair, tissue and cellular stimulation to increase blood circulation and stimulate hair growth, and to space the laser modules an appropriate distance way from the body region being treated.

In one embodiment, the functionality of the lasers is controlled by a microcontroller that pulses the lasers according to several preprogrammed pulsing schemes at various power levels. By programming the pulsing intervals of the lasers and the operating voltage to the lasers modules, the current drain and hence the power dissipation is reduced, thereby extending the battery life of the laser device, while still providing the desired stimulation and treatment.

In another embodiment, the laser device has a modular and removable head assembly that the user may interchange. In this way, the laser device can provide LLLT in a variety of different wavelengths of light (WOL), power and pulse duration's to treat conditions other applications other than hair loss. In other words, to stimulate hair growth the wavelength of the laser energy is typically within the range of 630-670 nanometers (nm). To treat the skin for wrinkle reduction the wavelength of the laser energy is generally in the 525-540 nm wavelength range. Different head assembly modules with a quick disconnect are therefore provided in a number of different wavelength ranges to accommodate a wide variety of treatments. For example, there may be assemblies directed to dermatological uses, skin rejuvenation, tanning, acne, hair removal, arthritis, pain, acupuncture, and massage. In alternative embodiments, the removable interchangeable head assembly modules may not have a specified wavelength range but may be a variety of specific wavelengths in the same head assembly module.

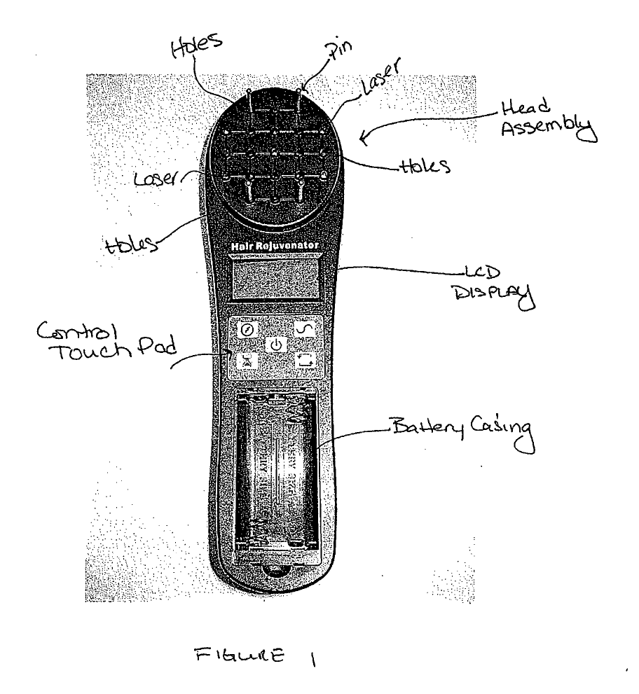

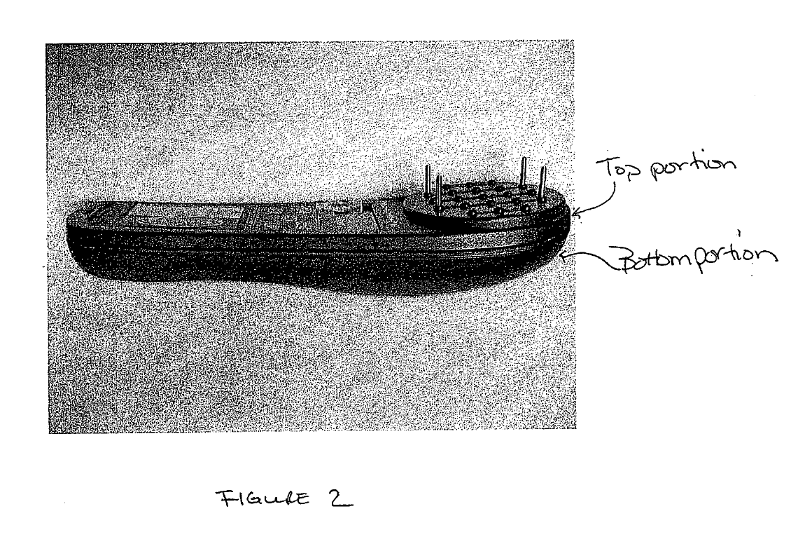

FIG. 1 illustrates an exemplary laser device having a head assembly, an LCD display, a control touch pad, and a battery compartment. The laser device housing, as best shown in FIG. 2, includes a top upper housing portion and a bottom lower housing portion. The head assembly, LCD display, battery compartment and control touch pad collectively represent the top upper portion of the housing structure. The bottom lower portion of the housing serves primarily as a cover to seal the internal components of the laser device. The housing is generally made of plastic; however, other rigid materials may be suitable.

Referring to FIG. 1, the head assembly includes a plurality of holes for mounting pins, sensors, lasers diodes and laser modules. In the embodiment of FIG. 1, there are four pins selectively positioned with respect to five lasers. However, any number of pin and laser combinations are contemplated. The pins in the head assembly are contractible compression spring type and mounted onto a head assembly printed circuit board. The pins are configured for additional tissue stimulation to increase blood circulation and also serve as a mechanism to regulate the distancing between the laser apertures and the body region being treated and also can be utilized as a sensory transducer. By maintaining the distance between the emanating aperture of the lasers and the body region subjected to treatment, the optimum amount of laser energy is appropriately distributed without maximizing the intensity of the laser energy and over-exposing the body region exposed to treatment. By selectively combining the pins and laser modules, a treatment region is defined wherein the laser energy is distributed to a substantially undisturbed portion of the body region being treated. More specifically, the pins and laser modules are mounted with respect to one another such that the pins never lead or follow the path of the laser beams. In an exemplary embodiment wherein the treatment application is hair growth stimulation and the region being treated is the head, the treatment region includes the hair, follicle, skin, scalp, and surrounding tissues, not just the scalp. Additionally, the pins may include ring magnets that magnetizes the pins for further stimulation while attracting unwanted debris and contaminates. In an exemplary embodiment wherein the treatment region is the head, the treatment region includes the hair, follicle, skin, scalp, and surrounding tissues, not just the scalp. The pins may also incorporate and induce a gentle electro pulse high voltage to the treatment area for additional stimulation. In addition sensory devices like themistor or thermal couples may be incorporated or connected to the metal pins for impedance, ohmic, thermo measurements.

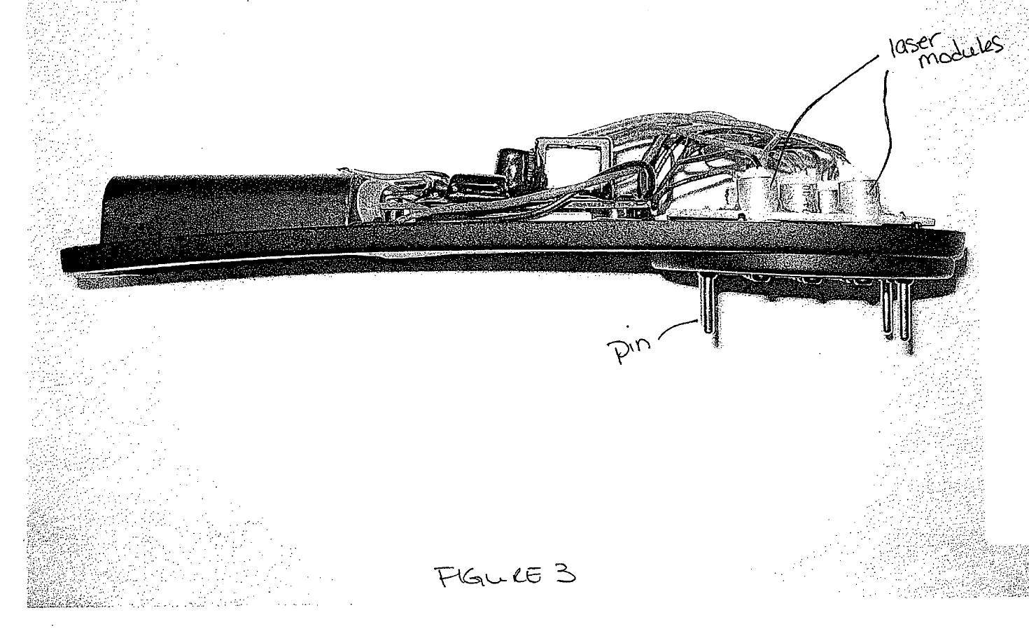

Each laser module in the head assembly includes a laser diode, a photo diode, a power control circuit, a lens, and a housing that are selectively mounted into the head assembly interchangeable module. The position of the laser modules is dependent in part on the number of laser diodes, laser modules, pins, motors and sensors that are installed in the head assembly module. FIG. 3 illustrates an exemplary side view of the laser device shown in FIGS. 1 and 2 illustrating the mounting configuration of the laser modules in the head assembly. The PCB platform on which the lasers, motors, sensors and pins are mounted is modular. In this way, the entire laser assembly can be easily interchanged or removed by the user. In one embodiment, the laser modules are mounted substantially perpendicular to the surface of the head assembly such that the laser energy is directed substantially downward. In another embodiment, the laser modules are mounted in the holes at a predetermined angle to effectively adjust the area of the treatment region.

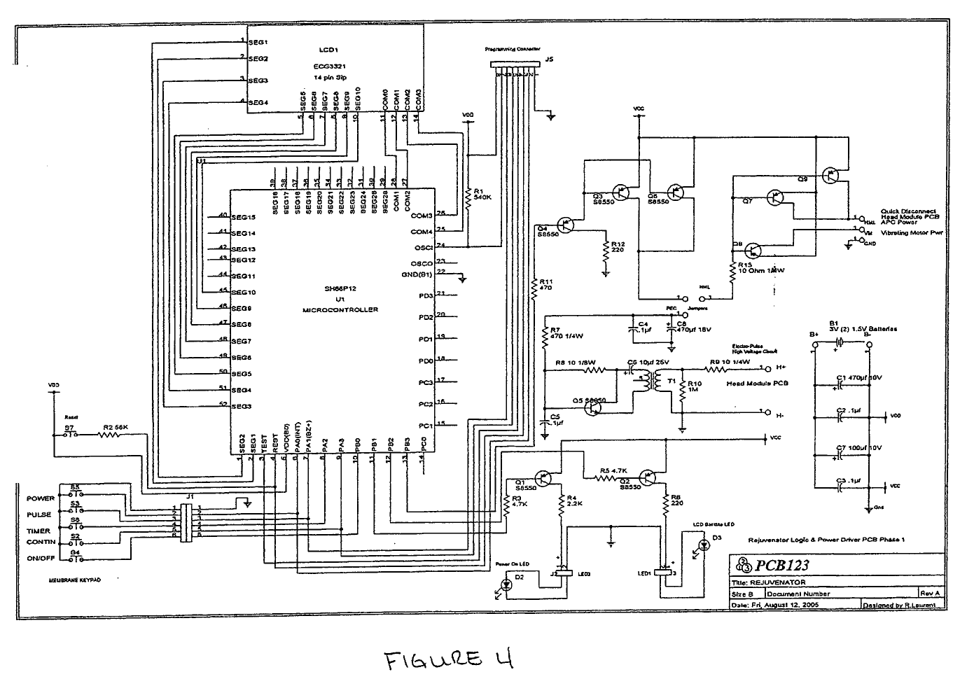

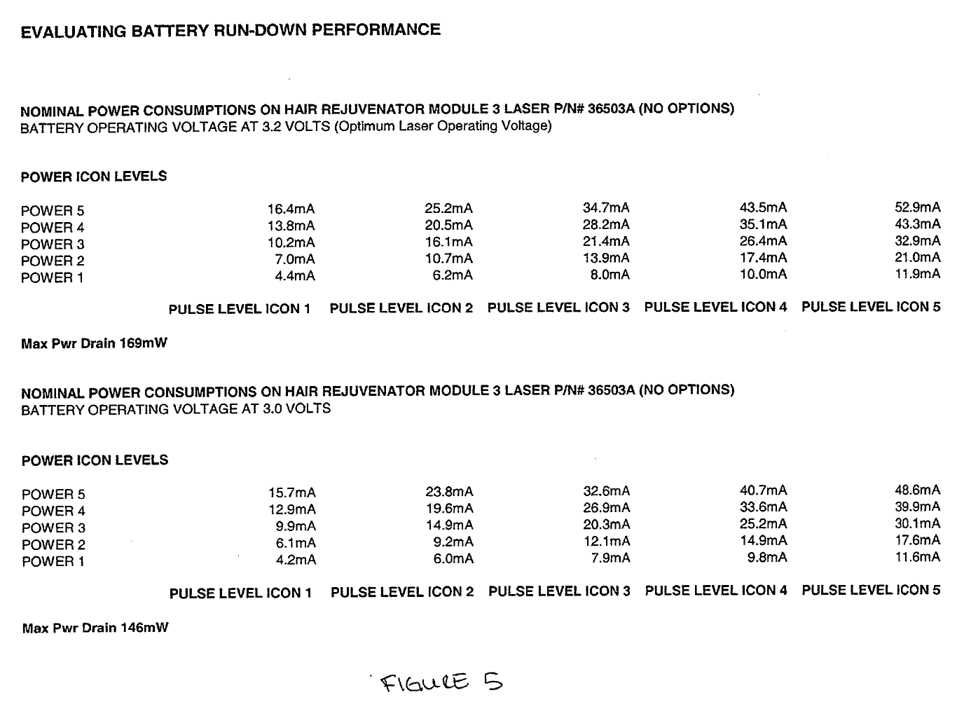

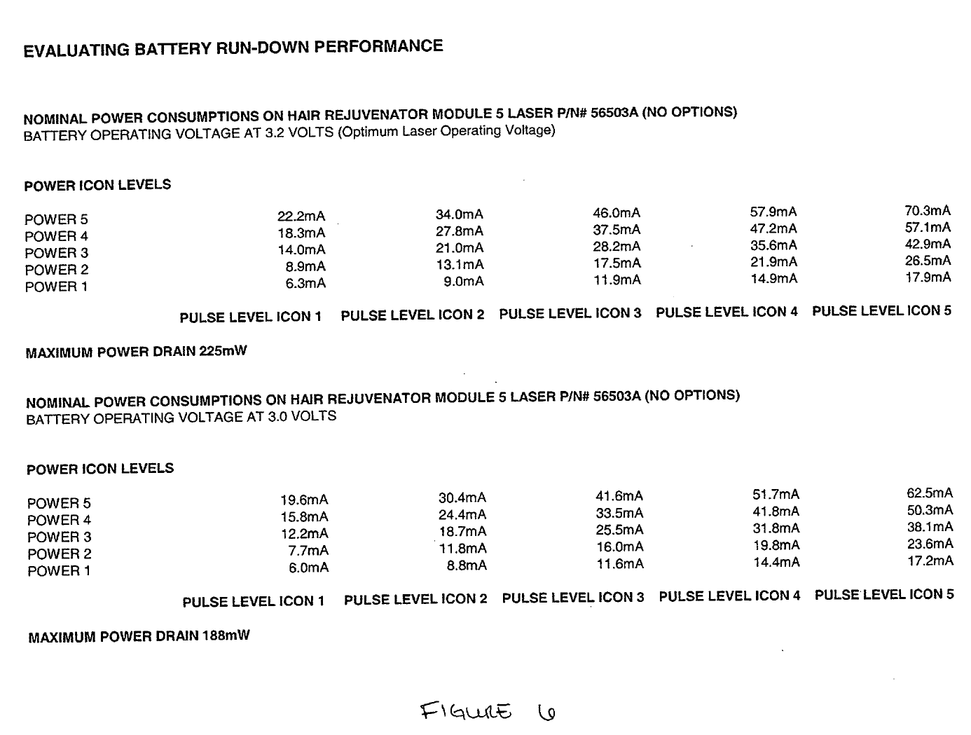

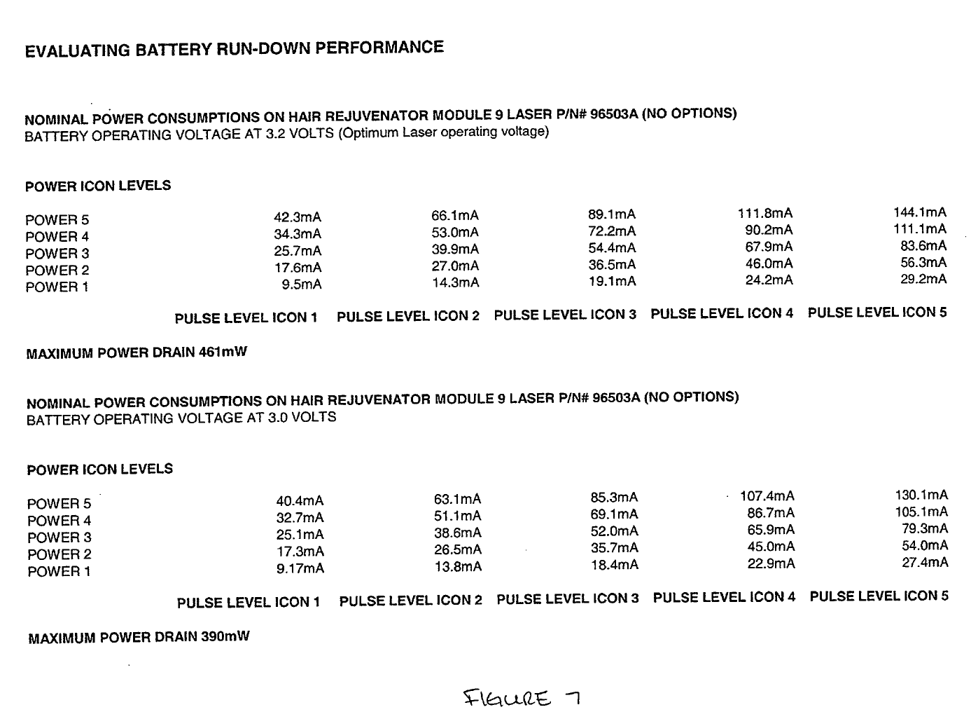

The overall functionality of the laser modules is controlled by an internal microcontroller that includes a CPU core, LCD driver, SRAM, timers, programmable ROM, alarm generators, oscillator, timer/counters with pre-diver circuits and I/O ports. The microcontroller is configured to selectively be programmed and controls the laser diodes, modules, motors, electro pulse voltage at different power densities and pulse intervals and durations. In this way, the overall voltage and current consumption is reduced, thereby extending the battery life of the laser device, yet still maximizing the laser energy for LLLT treatment in a pulsed mode. FIG. 4 illustrates an exemplary schematic of the device and the corresponding peripheral components. Additionally, FIGS. 5-7 illustrate the power consumption for exemplary laser devices having 3, 5, and 9 laser modules per device, respectively. Similarly, Exhibits A and B illustrate a battery power consumption matrix detailing the power consumption of a laser device having three and five laser modules, respectively. Further, a motor circuit may be implemented into the laser device that vibrates the contractible pins to massage the tissue for additional blood circulation and to loosen debris and contamination from clogged pores and hairfollicles.

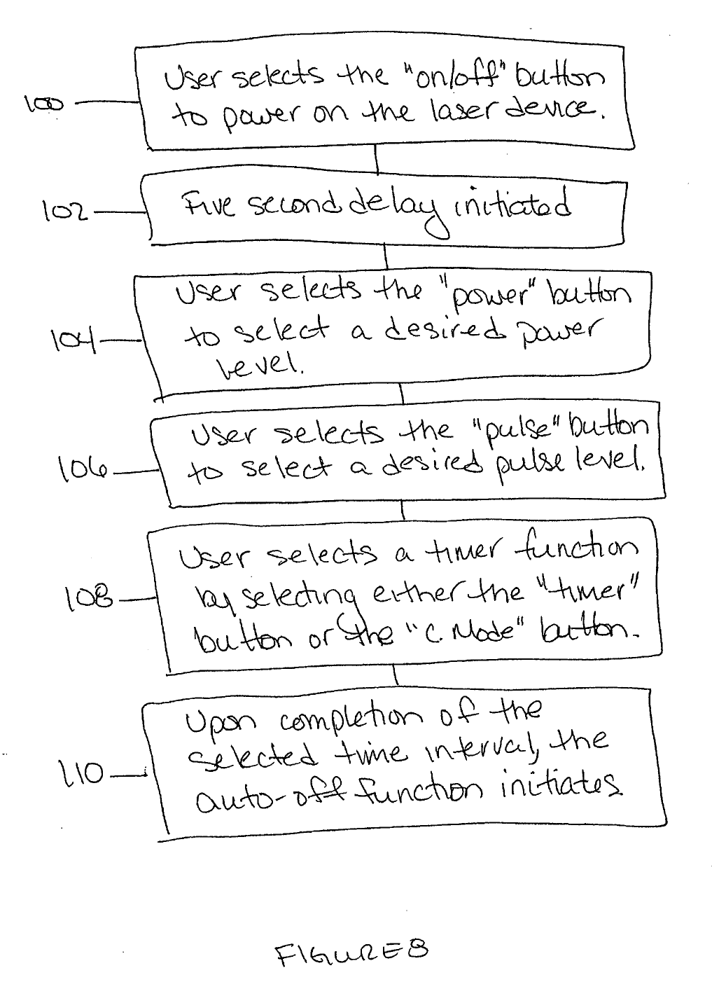

Functionally, the laser device operates in accordance with the flow chart of FIG. 8. In step 100, the power on/off button on the control touch pad is selected to turn on the laser device. In conjunction, a light emitting diode (LED) power “on” indicator on the upper housing (top) of the laser device turns on providing a visual verification that the laser device is active. In addition, selecting the power on/off button powers the LCD display. Once the power has been turned on in step 100, an approximate five second delay is initiated at step 102 while the laser device awaits a power level, pulse level, and time mode selection from the user. Generally, the user has five power levels and five pulse levels from which to select. With respect to a time interval, the user may select either a continuous time mode for up to an hour, or a five, ten or fifteen minute time interval settings. If the approximately five second delay expires without a selection being made, the laser device defaults to the last known selection. The order in which a power, pulse, or time selection is made is not germane to the functionality of the laser device.

At step 104, the user selects a power level by selecting the “power” button on the control touch pad. The power levels are pre-programmed into the microcontroller and may be dependent on the number of laser modules in a specific laser device. In an exemplary device, however, power level one is programmed at 2.4 volts. Each additional power level represents an incremental increase with respect to power level one. In one embodiment, the incremental increase per power level is 100 millavolts. One of ordinary skill in the art understands that the incremental power levels, as well as the base power level one is a variable that may be adjusted depending on the specifications of the application and the laser device.

At step 106, the user selects a pulse level by selecting the “pulse” button at the control touch pad. The pulse level in general represents the number of laser pulses and the pulse duration applied to the treatment region per second. In an exemplary embodiment, pulse level one is programmed to two pulses per second. For each increase in pulse level, the number of pulse per second is increased by one. For example, pulse level two represents three pulses per second and pulse level three represents four pulses per second. Similar to the power level settings, one of ordinary skill in the art understands that the pulse level settings and the pulse duration are adjustable and programmable at the factory and dependent on the specific laser device application. In addition, the frequency of the laser pulses may be modified by adjusting the pulse width duration or by modulating the pulse width. Pulsing the lasers modules advantageously reduces thermal runaway and power consumption of the laser modules thereby contributing to the extended battery life of the laser device. In addition, by pulsing the laser modules the cells, tissues, hair papilla, follicles and hair are allowed to rest and resonate, which has been shown to increase the effects of the cellular stimulation, which aids and promote cellular regeneration and hair growth.

At step 108, the user selects a timer function by selecting either the “timer” button or the “C.Mode” button on the control touch pad. By selecting the “timer” button, the user is able to select a five, ten or fifteen minute operation time intervals. In other words, if the user selects five minutes, the laser device will operate at the selected power and pulse level for five minutes. By selecting the “C.Mode” button, the user is able to customize the operation time interval; however, the maximum allotted treatment time is sixty minutes.

At the conclusion of a selected time interval, the auto off function initiates at step 110. This function begins timing at the completion of a selected time interval and automatically turns off the laser device after a five minute inactive period. This inactive time period may vary and is programmable at the factory with a warning alarm and may be adjusted depending on the specific laser device needs and applications.

The appendices (Appendix A and Appendix B) include exemplary functionality, design and specifications information related to a current prototype of the hand-held laser device disclosed herein and are a part of this disclosure.

While the present invention has been particularly shown and described with reference to the foregoing preferred embodiment, it should be understood by those skilled in the art that various alternatives to the embodiments of the invention described herein may be employed in practicing the invention without departing from the spirit and scope of the invention as defined in the following claims. It is intended that the following claims define the scope of the invention and that the method and system within the scope of these claims and their equivalents be covered thereby. This description of the invention should be understood to include all novel and non-obvious combinations of elements described herein, and claims may be presented in this or a later application to any novel and non-obvious combination of these elements. The foregoing embodiment is illustrative, and no single feature or element is essential to all possible combinations that may be claimed in this or a later application. Where the claims recite “a” or “a first” element of the equivalent thereof, such claims should be understood to include incorporation of one or more such elements, neither requiring nor excluding two or more such elements.

What is claimed is:

1. A hand-held modular laser device for stimulating a body region to promote hair growth, comprising:
a housing structure having a removable head assembly;
a plurality of pins configured to stimulate the body region;
a plurality of laser modules configured to provide laser energy to the body region; and
a microcontroller configured to control said plurality of laser modules by pulsing said plurality of laser modules at a selected power, pulse levels and pulse durations;
wherein said plurality of pins and said plurality of lasers are mounted through holes in said housing structure.
